(19) United States
(12) Patent Application Publication
(10) Pub. No.: US 2004/0030176 A1
(43) Pub. Date: Feb. 12, 2004

(54) Gamma hydroxy carbamate compounds and method of making and using the same

(76) Inventor: Walter H. Ohrbom, Hartland Township, MI (US)

Correspondence Address: BASF CORPORATION, ANNE GERRY SABOURIN, 26701 TELEGRAPH ROAD, SOUTHFIELD, MI, 48034-2442, US

(21) Appl. No.: 10/631,183

(22) Filed: Jul. 31, 2003

Related U.S. Application Data
(63) Continuation-in-part of application No. 09/998,365, filed on Nov. 29, 2001.
(63) Continuation-in-part of application No. 09/763,855, filed on Sep. 5, 2001.
(63) Continuation-in-part of application No. 10/285,634, filed on Oct. 31, 2002.
(63) Continuation-in-part of application No. 10/285,600, filed on Oct. 31, 2002.
(63) Continuation-in-part of application No. 10/285,594, filed on Oct. 31, 2002.
(63) Continuation-in-part of application No. 10/325,328, filed on Dec. 20, 2002.
Application No. 10/325,328 is a continuation-in-part of application No. 10/325,301, filed on Dec. 20, 2002.

(51) Int. Cl. C07C 271/20

(52) U.S. Cl. 560/158

(57) ABSTRACT

The invention provides gamma hydroxy primary carbamate functional compounds, film-forming compositions comprising such compounds and methods of making the same. The film-forming compositions disclosed herein comprise (I) a gamma hydroxy primary carbamate comprising one or more structures of the formula:

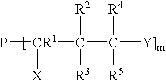

wherein X and Y are either a primary carbamate group or a hydroxyl group but are not the same, m is a number from 2 to 50, R¹, R², R³, R⁴, and R⁵ are each at least one of H, an alkyl group, a heteroatom containing group, and mixtures thereof, and P is at least one hydrocarbon based member selected from a compound, an oligomer or polymer having more than 6 carbon atoms.

### FIELD OF THE INVENTION

This application claims the benefit of prior U.S. patent application Ser. No. 09/998,365, filed Nov. 29, 2001, Ser. No. 09/763,855, filed Feb. 27, 2001, Ser. No. 10/285,634, filed Oct. 31, 2002, Ser. No. 10/285,600, filed Oct. 31, 2002, Ser. No. 10/285,594, filed Oct. 31, 2002, Ser. No. 10/325,328, filed Dec. 20, 2002, and Ser. No. 10/325,301, filed Dec. 20, 2002.

### BACKGROUND

The invention relates to gamma hydroxy primary carbamate functional materials, compositions incorporating such materials and methods of making the same.

### BACKGROUND

Curable coating compositions, especially thermoset coatings, are widely used in the coatings art. They are often used for topcoats in the automotive and industrial coatings industry.

High-gloss and color-plus-clear composite coatings are particularly useful as topcoats where exceptional gloss, depth of color, distinctness of image, or special metallic effects are desired. The automotive industry has made extensive use of these coatings for automotive body panels. These coatings require an extremely high degree of clarity and a low degree of visual aberrations at the surface of the coating in order to achieve desired visual effects such as a high distinctness of image (DOI).

As a result, high-gloss and composite color-plus-clear coatings are susceptible to a phenomenon known as environmental etch. Environmental etch manifests itself as spots or marks on or in the finish of the coating that often cannot be rubbed out. It can be difficult to predict the degree of resistance to environmental etch that a high gloss or color-plus-clear composite coating will exhibit. Many coating compositions known for their durability and/or weatherability when used in exterior paints, such as high-solids enamels, do not provide the desired level of resistance to environmental etch when used in high gloss coatings and color-plus-clear composite coatings.

Many compositions have been proposed for use as the clearcoat portion of color-plus-clear composite coating systems, such as polyurethanes, acid-epoxy systems and the like. However, many prior art systems suffer from disadvantages such as coatability problems, compatibility problems with the pigmented basecoat, and/or solubility problems. Moreover, very few one-pack coating compositions have been found that provide satisfactory resistance to environmental etch, especially in the demanding environment of automotive coatings.

It has been found that carbamate functional polymers such as those described in U.S. Pat. No. 5,356,669 can be used to provide coating compositions which exhibit significantly improved environmental etch resistance. Carbamate functional polymers have been used to provide commercially advantageous coatings compositions, especially as clearcoats in composite color-plus-clear coatings.

Unfortunately, some carbamate functional compounds and/or polymers known in the prior art are vulnerable to instability and decomposition, especially with respect to the formation of cyclic carbonates and carbamates. This results in difficulties in manufacturing and storage.

In addition, although coating compositions containing carbamate functional polymers generally provide the performance properties currently required by the automotive industry, continuous improvement is always desired. As a result, it would be advantageous to provide improvements in solids or % nonvolatile, flexibility, scratch & mar resistance, cold crack resistance, chip resistance and/or the like. At the same time, such improvements must be achieved without any decrease in environmental etch resistance or other commercially required performance properties, including processability and stability.

Improvements with respect to hydroxy alkyl carbamates are especially desirable. Hydroxy alkyl carbamates are advantageous because they can have two different reactive functional groups on a single pendant group, i.e., a hydroxy group and a carbamate group. Ideally, the dual reactive functional groups are close enough to affect a synergistic effect upon each other with respect to increased reactivity. For example, some beta hydroxy carbamates show improved water solubility as disclosed in U.S. Pat. No. 6,346,591. However, some prior art hydroxy alkyl carbamates have shown an undesirable instability, particularly when exposed to higher temperatures and/or aqueous environments.

It would be therefore be desirable to provide hydroxy alkyl carbamates having improvements in both reactivity and stability and applicable for use in a wide variety of coating compositions. Desired applications include primers, basecoats, clearcoats, two-component systems, anti-chip coating compositions, water borne coatings, solvent borne coatings, coatings for flexible substrates, powder coatings, solventless powder-slurry coatings, solventless liquid coatings, and the like. It would be particularly advantageous to provide hydroxy alkyl carbamates that could be used in those coating compositions traditionally subjected to challenging stability conditions such as powder coating extrusion processes.

The prior art has failed to address and rectify these issues.

The preparation of monocarbamate alcohols by the ammonolysis of cyclic carbonates prepared from substituted propanediols is disclosed in Some Anticonvulsant Agents Derived from 1,3-Propanediols, Ludwig, B. J. and Piech, E. C.; J. Am Chem. Soc. (1951) 73 5779-81. CAN 47:3228.

U.S. Pat. No. 5,719,237, Rehfuss et al., discloses the use of carbamate functional compounds (a) having a plurality of carbamate groups prepared by a transcarbamylation reaction wherein an alcohol or hydroxylalkyl carbamate is reacted with an alkyl carbamate. The '237 patent teaches that it is desirable to avoid the inclusion of hydroxyl groups in compound (a) as such hydroxyl groups lead to the formation of vulnerable ether bridges.

U.S. Pat. No. 5,907,024, Ohrbom et al., and U.S. Pat. No. 5,945,499 disclose the use of hydroxyalkyl carbamates of the general structure

—C(OH)—(CH₂)n—O—C(O)—NHR

wherein n is an integer from 0 to 6 and R is H or an alkyl group of from 1 to 4 carbons.

U.S. Pat. No. 5,760,127, Bammel et al, and U.S. Pat. No. 6,262,297, Clements et al., disclose hydroxyalkylcarbamates produced by the reaction of anhydrous ammonia or aqueous ammonium hydroxide with a six-membered cyclic carbonate. Bammel et al discloses that five-membered rings are preferred, not as a result of better performance, but as a result of their ease of synthesis and greater degree of commercial availability. Clements et al teaches that six-membered rings are preferred due to increased stability. However, the cost and commercial availability of the six-membered cyclic carbonates renders the process and resultant products to be less than cost effective. Also, depending on the location of any substituent groups on the starting cyclic carbonate, the process disclosed in Clements produces a reaction product which is a compound comprising a mixture of structures with varying reactivity and selectivity. Finally, Clements does not disclose that the hydroxy alkyl carbamates can be used in film-forming coating compositions. Rather, Clements teaches only that the hydroxyl site is suitable for making carbamate functional polymers.

WO 0156978, Rink, et al discloses positionally isomeric diethyloctanediol dicarbamates and diethyloctanediols diallophanates. The dicarbamate and diallophanate species have no hydroxyl functionality and are made from position isomers of diethyloctane diols.

The prior art has failed to provide hydroxy alkyl carbamate functional materials that possess improved stability with respect to decomposition and the formation of undesirable cyclic carbonates and carbamates.

It is therefore desirable to provide hydroxy alkyl carbamates having improved stability and reactivity. It would be particularly advantageous to provide hydroxy alkyl carbamates that have the increased water solubility and reactivity of beta hydroxy carbamates as well as improved thermodynamic and water stability.

### SUMMARY OF THE INVENTION

Disclosed herein is a gamma hydroxy primary carbamate comprising one or more structures of the formula:

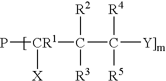

wherein X and Y are either a primary carbamate group or a hydroxyl group but may not be the same, m is a number from 2 to 50, R¹, R², R³, R⁴, and R⁵ are each at least one of H, an alkyl group of from one to four carbons, a heteroatom containing group, or mixtures thereof, and P is at least one hydrocarbon based member selected from a compound, an oligomer or polymer having more than 6 carbon atoms.

Also disclosed herein is a film-forming composition comprising (1) a gamma hydroxy primary carbamate comprising one or more structures of the formula:

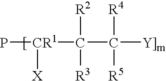

wherein X and Y are either a primary carbamate group or a hydroxyl group but are not the same, m is a number from 1 to 50, R¹, R², R³, R⁴, and R⁵ are each at least one of H, an alkyl group of from one to four carbons, a heteroatom containing group, or mixtures thereof, and P is at least one hydrocarbon based member selected from a compound, an oligomer or polymer having more than 6 carbon atoms.

The invention also provides a method of making a gamma hydroxy primary carbamate functional material, comprising providing a material P comprising two or more cyclic carbonate groups (bi) and of the structure:

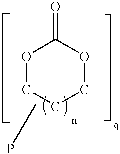

wherein n is 1, q is a number from 2 to 50, and P is a hydrocarbon based material selected from the group consisting of compounds, oligomers, and polymers having more than 6 carbon atoms, and reacting at least one cyclic carbonate functional group (bi) with ammonia to provide a gamma hydroxy primary carbamate comprising one or more structures of the formula:

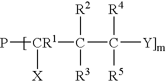

wherein X and Y are either a primary carbamate group or a hydroxyl group but may not be the same, m is a number from 2 to 50, R¹, R², R³, R⁴, and R⁵ are each at least one of H, an alkyl group, a heteroatom containing group, or mixtures thereof, and P is at least one hydrocarbon based member selected from a compound, an oligomer or polymer having more than 6 carbon atoms.

The above described features and others are exemplified by the following detailed description.

### DETAILED DESCRIPTION OF THE INVENTION

The disclosed gamma hydroxy primary carbamates comprise one or more structures of the formula:

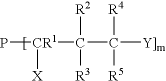

wherein X and Y are either a primary carbamate group or a hydroxyl group but may not be the same, m is a number from 2 to 50, R¹, R², R³, R⁴, and R⁵ are each at least one of H, an alkyl group, a heteroatom containing group, or mixtures thereof, and P is at least one hydrocarbon based member selected from a compound, an oligomer or polymer having more than 6 carbon atoms.

It will be appreciated that the phrase “gamma hydroxy primary carbamate group” refers to functional groups of the structure:

NH₂—C(O)—O—C′—C′ₙ—C′(OH)

wherein C′ is a saturated carbon having substituents selected from hydrogen, alkyl groups, groups containing heteratoms such as oxygen, nitrogen, silane, and the like, and combinations thereof, and n is 1. However, although the substituents for C′ may contain heteroatoms as discussed below, such substituents may not include beta hydroxy groups, i.e., hydroxy groups that are attached to a carbon that is adjacent to a carbon atom attached to a primary carbamate group. All hydroxy groups present in the gamma hydroxy primary carbamate functional group must be at least gamma or higher, i.e., there must be one or more carbons between the carbon to which the primary carbamate group is attached and the carbon to which the hydroxy group is attached.

The term “structures” as used herein refers to isomers that satisfy the requirements of the instant invention. “Isomers” as used herein refers to structural and position isomers that have the same empirical chemical formula. “Structures” refers to those isomers which have the same empirical chemical formula but which satisfy the requirements of the instant formula. For the purposes of the instant invention, it will be appreciated that a single material may comprise one or more than one structure. Illustrative examples of structural isomers are 2-ethyl-1,3-hexanediol and 2-propyl-1,3-pentanediol. Illustrative examples of position isomers are 2-ethyl-1,3-hexanediol and 2-ethyl-1,4-hexanediol. Illustrative examples of isomers which are both structural and position isomers are 2-ethyl-1,3-hexanediol and 2-propyl-1,4-pentanediol.

In general, the gamma hydroxy primary carbamate functional material of the invention may comprise one or more structures that satisfy the above requirements. In another embodiment, the reactive compound may comprise two or more structures that are isomerically different as defined above but which each satisfies the above noted requirements.

X and Y may be either a primary carbamate group or a hydroxyl group but may not be the same. That is, X and Y may not be the same in a single or particular pendant group. However, as discussed above, the gamma hydroxy primary carbamate functional materials of the invention will have at least two and up to fifty pendant groups of the structure:

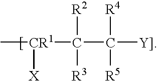

It will therefore be appreciated that X may be hydroxyl in some pendant groups and primary carbamate in other pendant groups. Similarly, Y may be hydroxyl in some pendant groups and primary carbamate in other pendant groups. It is therefore within the scope of the invention that within the same molecule, some X's and Y's are both primary carbamate while some X's and Y's are both hydroxyl, so long as X and Y are different with respect to a single pendant group defined by the above pendant group structure.

As used herein, “primary carbamate group” refers to the functional group having the structure

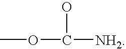

Thus, the primary carbamate group of the invention may be defined as a terminal or pendent primary carbamate group.

Hydroxyl groups suitable for use as X or Y include primary, secondary and tertiary hydroxyl groups. It will be appreciated that whether the single OH group is primary, secondary or tertiary depends on whether the OH group represents X or Y as well as the identity of substituents R¹, R², R³, R⁴ and R⁵.

For example, if Y is hydroxyl, the hydroxyl group will be primary when R⁴ and R⁵ are hydrogen. If Y is hydroxyl, and one of R⁴ and R⁵ is hydrogen and the other nonhydrogen as defined below, the hydroxyl will be secondary. If both R⁴ and R⁵ are not hydrogen and are as defined below, the hydroxyl group Y will be tertiary.

If X is hydroxyl, the hydroxyl group will never be primary and may only be secondary if R¹ is hydrogen. If R¹ is not hydrogen, the hydroxyl group X will be tertiary.

In one preferred embodiment, the hydroxyl group will be a primary or secondary hydroxyl group. Thus, in some preferred embodiments, X will be hydroxyl and R¹ will be hydrogen. In some other preferred embodiments, Y will be hydroxyl and at least one of R⁴ and R⁵ will be hydrogen. In one particularly preferred embodiment, Y will be hydroxyl and both R⁴ and R⁵ will be hydrogen.

In general, R, R², R³, R⁴ and R⁵ may be H, an alkyl group, a heteroatom containing group, or mixtures thereof. In general, the compounds of the invention are particularly suited for use in water-soluble and water dispersible compositions. As a result, it is particularly desirable to optimize the water solubility of the gamma hydroxy primary carbamate groups of the invention. In one preferred embodiment, R¹, R², R³, R⁴ and R⁵ will be selected from hydrogen, alkyl groups having from one to four carbons, and mixtures thereof. In one especially preferred embodiment, at least some of R¹, R², R³, R⁴ and R⁵ will be hydrogen. In another particularly preferred embodiment, all of R¹, R², R³, R⁴ and R⁵ will be hydrogen

Illustrative alkyl groups suitable for use as one or more of R¹, R², R³, R⁴ and R⁵ may have from 1 to 12 carbons, preferably 1 to 7, more preferably 1 to 4 and most preferably 1 to 2 carbons. Suitable groups include pentyl groups, octyl groups, hexyl groups, heptyl groups, methyl groups, ethyl groups, propyl groups, butyl groups and mixtures thereof. Such groups may be linear or branched. As used herein, the term “branched” refers to both lateral branches and forked branches. Lateral refers to a branch of two small chains at the end atom of a carbon chain. Forked refers to a branch of two small chains in the middle of a carbon chain. Any individual substituent R¹, R², R³, R⁴ and R⁵ may have both branching and forking therein. However, in one preferred embodiment, no more than two of R¹, R², R³, R⁴ and R⁵ will have alkyl groups of more than four carbons. In addition, it is within the scope of the invention for two or more of the various R substituents to be connected with each other.

In one preferred embodiment, one or more of R¹, R², R³, R⁴ and R⁵ will be selected from alkyl groups having from one to four carbons. Illustrative groups include methyl groups, ethyl groups, propyl groups, butyl groups and mixtures thereof. In one more preferred embodiment, any alkyl group used as one or more of R¹, R², R³, R⁴ and R⁵ will have no more than two carbon atoms. In one most preferred case, alkyl groups of one carbon, i.e., methyl groups, will be used as one or more of R¹, R², R³, R⁴ and R⁵.

Illustrative heteroatom containing groups suitable for use as one or more of R¹, R², R³, R⁴ and R⁵ include ether groups formed from alkylene oxides, urea and cyclic urea groups, amide groups, ester groups, acid groups, primary carbamate groups, hydroxyl groups that are at least gamm relative to a primary carbamate group, and mixtures thereof. Illustrative alkylene oxide groups include polyethylene oxide groups, ethylene urea groups, polylactone groups, and the like.

However, although it is within the scope of the invention for any one or more of substituents R¹, R², R³, R⁴ and R⁵ to be a heteroatom containing group, it is most preferred that the majority of heteroatom containing groups be contained within P as discussed below. While not wishing to be bound to a particular theory, it is believed that the presence of heteroatom containing groups in the gamma hydroxy primary carbamate portion of the molecule contributes to increased water solubility. Such increased water solubility, although generally favored, is less desirable when located close to the two reactive groups of the gamma hydroxy primary carbamate group that will react with the crosslinking agent(s) of a thermoset coating composition. What is desired is a film linkage or crosslinking site that is not adjacent to functional groups contributing to increased water solubility. Thus, in one more preferred embodiment, the substituents R¹, R², R³, R⁴ and R⁵ will be substantially free of any heteroatom containing groups on average per molecule.

The subscript m may be a number of about 1 to about 50, preferably about 2 to about 50, more preferably about 4 to about 30, and most preferably about 4 to about 10. In one preferred embodiment, m will be at least 4.

In general, material P is a hydrocarbon-based material that may or may not contain heteroatoms. Material P may be a compound, oligomer, polymer, or a mixture thereof. Material P may be aliphatic, cycloaliphatic, aromatic, unsaturated, saturated, and mixtures thereof and may have a number average molecular weight of about 1 to about 1,000,000 Daltons. More preferably, material P may have a number average molecular weight of about 15 to about 50,000 Daltons, most preferably about 174 to about 8,000 Daltons. Also, it has been found that it is advantageous when P at a minimum contains more than 6 carbons, preferably more than 8 carbon atoms and most preferably more than 10 carbon atoms.

In one embodiment, material P may, and most preferably will, contain one or more heteroatoms or heteroatom containing groups. “Heteroatoms” as used herein refers to atoms other than carbon or hydrogen. Preferred heteroatoms are O, N, Si, and mixtures thereof. Such heteroatom containing groups may be pendent, terminal or within the body of P. It will be appreciated that heteroatoms or heteroatom containing functional groups may used to impart particular performance characteristics such as water solubility (i.e., hydroxyl groups or acid groups) or adhesion (i.e., epoxy groups). Alternatively, some heteroatom containing groups may be intended for reaction with one or more crosslinking agents (B).

Examples of groups containing heteratoms include hydroxyls, ethers, esters, amids, ureas, urethanes, silanes, epoxy groups, primary carbamate groups, acid groups, and mixtures thereof. Preferred heteroatom containing groups are hydroxyls, ethers, esters, urethanes, acid groups, epoxy groups, and mixtures thereof. Functional groups contributing to increased water solubility or water dispersiblity are especially preferred, such as acid groups, amide groups, hydroxyl groups, mixtures thereof, and the like.

“Polymer” as used herein refers to materials having at least ten repeating units, more preferably greater than 10 repeating units. The term “repeating units” as defined as herein refers to groups of atoms that are the reaction product result or residue of the reaction of two or more monomers. Such repeating units will generally have an individual number average molecular weight in the range of about 28 to about 750 Daltons.

In general, polymers suitable for use as material P will have a number average molecular weight in the range of about 1500 to about 1,000,000 Daltons, preferably about 1500 and about 50,000 Daltons, most preferably about 1500 to about 15,000 Daltons.

For the purposes of the instant invention, the term “oligomer” refers to materials having from two to nine repeating units or mixtures of repeating units. In general, suitable oligomers for use in the instant invention will have number average molecular weights in the range of about 202 to about 1499 Daltons. It will be appreciated by those of skill in the art that because oligomers and polymers are both based on repeating units of monomeric materials; high molecular weight oligomers may overlap the low molecular weight end range for polymers.

“Compounds” as used herein refers to materials that do not contain two or more of the same repeating units. In general, compounds may have number average molecular weights in the range of about 450 or less, more preferably less than about 175 and more preferably less than about 144. In another embodiment, compounds may have a number average molecular weight of about 1 to about 450, more preferably about 1 to about 175, and most preferably about 15 to about 144.

Compounds suitable for use as P include hydrogen, alkyl groups, mono or polyfunctional compounds such as nonchain-extended aliphatics, cycloaliphatics, aromatics which may or may not contain heteroatoms, and the like, as well as mixtures thereof. For example, P may be hydrogen as indicated in the structure below:

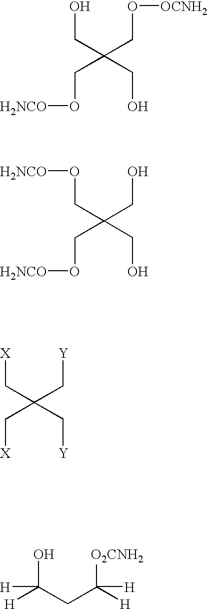

It will be appreciated that in these structures, one or more of substituents R¹, R², R³, R⁴ and R⁵ contain hydrogen, alkyl groups, heteroatom containing functional groups, or mixtures thereof, while X and Y are as defined above.

While P may be a compound, an oligomer, a polymer or a mixture thereof, P will most preferably be a polymer and/or oligomer.

Examples of suitable oligomers and/or polymers useful as material P include the following: biurets and isocyanurates, homopolymers of diisocyanate materials such as isocyanurates, acrylic, modified acrylic, polyurethane, polyester, polylactones, polyurea, alkyd, polysiloxane, polyethers, epoxy upgrades, mixtures thereof, and the like. Oligomers and polymers preferred for use as material P are polyurethane, polyester, acrylic, and the like. Most preferred polymers and oligomers for use as material P are polyurethanes, acrylics and isocyanurates.

In one embodiment, P may be an acrylic. Suitable acrylic polymers may have a molecular weight of about 1500 to about 1,000,000, and more preferably of about 1500 to about 50,000. As used herein, “molecular weight” refers to number average molecular weight, which may be determined by the GPC method using a polystyrene standard. Such polymers are well-known in the art, and can be prepared from monomers such as methyl acrylate, acrylic acid, methacrylic acid, methyl methacrylate, butyl methacrylate, cyclohexyl methacrylate, styrene, maleic anhydride, and the like as discussed below.

Modified acrylics can also be used as the material P according to the invention. Such acrylics may be polyester-modified acrylics or polyurethane-modified acrylics, as is well known in the art. Polyester-modified acrylics modified with ε-caprolactone are described in U.S. Pat. No. 4,546,046 of Etzell et al, the disclosure of which is incorporated herein by reference. Polyurethane-modified acrylics are also well known in the art. They are described, for example, in U.S. Pat. No. 4,584,354, the disclosure of which is incorporated herein by reference. A non-limiting example of one such polymer is an acrylic resin made up of hydroxyethyl methacrylate, methyl methacrylate, and butyl acrylate which is then half capped with a diisocyanate such as isophorone diisocyanate to make an isocyanate functional polymer useful as material P.

Polyesters and ester oligomers can also be used as P. Such polyesters are well-known in the art, and may be prepared by the polyesterification of organic polycarboxylic acids (e.g., phthalic acid, hexahydrophthalic acid, adipic acid, maleic acid) or their anhydrides with organic polyols containing primary or secondary hydroxyl groups (e.g., ethylene glycol, butylene glycol, 1,6-hexanediol, neopentyl glycol).

P may optionally comprise one or more additional functional groups (bii) that may or may not contain heteroatoms. Such additional functional groups (bii) may be defined as any reactive functional group that is essentially inert with respect to cyclic carbonate groups under the reaction conditions used to ring open a cyclic carbonate functional group. In one preferred embodiment, optional functional groups will be reactive with a reactive functional group of a curing agent (B). Illustrative examples of optional functional groups (bii) include blocked isocyanates, hydroxy, aminoplast, ethylenically unsaturated groups, primary carbamate groups, and the like.

Also provided herein is a method of making the disclosed gamma hydroxy primary carbamate functional materials. The method comprises providing a starting material comprising two or more cyclic carbonate groups (bi) and of the structure:

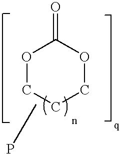

wherein n is 1, q is a number from 2 to 50, and P is a hydrocarbon based material selected from the group consisting of compounds, oligomers, and polymers having more than 6 carbon atoms, and reacting at least one cyclic carbonate functional group (bi) with ammonia to provide a gamma hydroxy primary carbamate group of the structure:

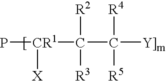

wherein X and Y are either a primary carbamate group or a hydroxyl group but may not be the same, m is a number from 2 to 50, R¹, R², R³, R⁴, and R⁵ are each at least one of H, an alkyl group, a heteroatom containing group, or mixtures thereof, and P is at least one hydrocarbon based member selected from a compound, an oligomer or polymer having more than 6 carbon atoms.

Starting materials suitable for use in the disclosed method are those materials that comprise two or more cyclic carbonate groups (bi) and are of the formula:

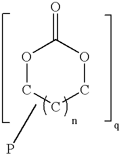

wherein P is as described above, q is a number from about 2 to about 50 and n is 1. Thus, illustrative cyclic carbonate groups (bi) are those cyclic carbonate groups having six-membered cyclic carbonate rings. The carbons in the above cyclic carbonate structure are fully saturated with the groups described above with respect to substituents R¹, R², R³, R⁴, and R⁵, i.e., either hydrogen atoms, alkyl groups, heteroatom containing groups, and mixtures thereof, except for the carbon atom linked to material P.

It will be appreciated that the required starting materials used in the disclosed method must have at least two cyclic carbonate groups, preferably more than two, and most preferably at least 4 cyclic carbonate groups on average per molecule of material P. Thus, while it is conceivable that there may be individual molecules that contain less than two cyclic carbonate groups, on average, each molecule will have two or more. In one embodiment, q will be about 4 to about 50, more preferably about 4 to about 20, and most preferably from about 4 to about 10, on average per molecule of material P.

When P is an acrylic polymer or oligomer, the required two or more cyclic carbonate functional groups of the starting material can be incorporated into the ester portion of an acrylic monomer.

In one embodiment of the invention, the starting material may result from the use of a monomer mixture (a) that is polymerized under polymerization conditions, especially free radical polymerization conditions, to make an acrylic oligomer or polymer backbone (b).

Monomer mixture (a) may comprise ethylenically unsaturated monomers having at least one carbon-carbon double bond that is reactive with another carbon-carbon double bond under conventional or controlled polymerization conditions. As used herein, ‘polymerization’ refers to oligomerization or polymerization reaction conditions wherein the temperature is between room temperature (approximately 20° C./68° F.) and no more than 180° C./356° F., more preferably from 70 to 140° C./158 to 284° F., and most preferably from 110 to 140° C./230 to 284° F. Such reaction conditions may relate to conventional polymerization reactions such as free radical polymerization as well as controlled or living polymerization reactions such as ATRP, and the like as discussed below.

In a preferred embodiment of the invention, polymerization as used herein refers to reaction conditions that are free of any catalysts that can activate an oxirane group. Illustrative examples of such oxirane activating catalysts are tertiary amine or quaternary salts (e.g., tetramethyl ammonium bromide), combinations of complex organotin halides and alkyl phosphonium halides (e.g., (CH₃)₃SnI, Bu4SnI, Bu4PI, and (CH₃)₄PI), potassium salts (e.g., K₂CO₃, KI) in combination with crown ethers, tin octoate, calcium octoate, and the like.

The most preferred polymerization techniques are free radical polymerizations that may take place in solvent or water but will most preferably take place in solvent. Illustrative examples of suitable organic solvents include aromatic solvents, ketone solvents, ester solvents, ether solvents, alcoholic solvents, and combinations thereof. In a preferred embodiment of the invention, free radical polymerization reaction conditions will be used which are free of catalysts such as Lewis acids and strong sulphonic acids having a pKa of less than 2.0.

In one preferred embodiment, free radical polymerization of ethylenically unsaturated monomers will take place in the presence of temperatures of about 80 to 140° C. an absence of any epoxy ring activating catalysts, and an absence of any water or alcohols that are reactive with cyclic carbonate functional groups under such temperatures. In one preferred embodiment, the monomer (ai) will be present in an amount sufficient to provide at least 4 cyclic carbonate groups per molecule on average. In other preferred embodiments, the oligomerization or polymerization conditions will be such that at least two cyclic carbonate functional groups per molecule on average remain inert, preferably at least about three or more cyclic carbonate functional groups on average per molecule, and most preferably about 4 to about 50 cyclic carbonate groups on average per molecule.

Alternatively, the monomer mixture comprising a cyclic carbonate functional ethylenically unsaturated monomer may be polymerized using controlled or living radical polymerization processes as described by Matyjaszewski and Krysztof in Chem. Reviews, Vol. 101 pg 2921-2990 (2001), or by iniferter process as described by Kuchanov, in J. of Polymer Science, Part A: Polymer Chemistry Vol 32 pg 1557-1568 (1994), and Gaofenzi Xuebao Vol 2 pg 127-136 (2002), nitroxide-mediated polymerization as described by Zaremski, in Russian Polymer News Vol 4 pg 17-21 (1999), and Wang, in Abstracts of Papers, 224th ACS National Meeting, Boston, Mass., United States, Aug. 18-22, 2002 (2002), all of which are incorporated by reference herein.

It is an aspect of this embodiment that monomer mixture (a) comprise a monomer (ai) having at least one cyclic carbonate group and the structure:

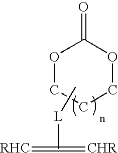

wherein L is a linking group selected from heteroatom containing groups such as ester groups, ether groups, urethane groups, urea groups; amide groups; aliphatic groups; cycloaliphatic groups; aromatic groups; and mixtures thereof, n is one, and R is either hydrogen or an alkyl group of from one to six carbons. For example a material such as 5-hydroxy-1,3-dioxan-2-one:

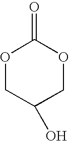

may be reacted with methacrylic anhydride to make 1,3-dioxan-2-one-5-methacrylate:

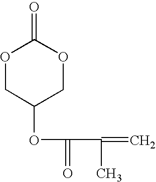

wherein L is an ester group. Alternatively, the hydroxyl functional carbonate can also be reacted with an isocyanate such as HDI or 2-isocyanatoethylacrylate. Illustrative hydroxy functional cyclic carbonates are commercially available and may be found in R. H. Wollenberg, U.S. Pat. No. 4,585,566 and E. J. Vandenberg, U.S. Pat. No. 6,300,458, hereby incorporated by reference.

Preferred groups suitable for use as linking group L are esters and urethanes, with esters being most preferred.

Monomer (ai) will be present in monomer mixture (a) in an amount of about 1 to about 100% by weight, based on the total weight of monomer mixture (a), more preferably about 5 to about 90%, and most preferably about 20 to about 70%, based on the total weight of monomer mixture (a). Those skilled in the art will appreciate that the requirement that material P contain on average at least two cyclic carbonate groups per molecular will necessitate the use of higher number average molecular weights for those oligomers and/or polymers of material P made from monomer mixtures having a low weight percent of monomer (ai).

Monomer (ai) can be prepared by the reaction of a glycidyl-group containing polymerization monomer with carbon dioxide to convert the oxirane group to a cyclic carbonate group. Examples of suitable oxirane group-containing polymerizable monomers include, without limitation, glycidyl acrylate, glycidyl methacrylate, glycidyl crotonate, and allyl glycidyl ether. This can be done at any pressure from atmospheric up to supercritical CO2 pressures, but is preferably under elevated pressure (e.g., 60-150 psi). The temperature for this reaction is preferably 60-150° C. Useful catalysts that activate the oxirane ring may be used. Illustrative catalysts include any that activate an oxirane ring, such as tertiary amine or quaternary salts (e.g., tetramethyl ammonium bromide), combinations of complex organotin halides and alkyl phosphonium halides (e.g., (CH₃)₃SnI, Bu₄SnI, Bu₄PI, and (CH₃)₄PI), potassium salts (e.g., K₂CO₃, KI) preferably in combination with crown ethers, tin octoate, calcium octoate, and the like.

Alternatively, cyclic carbonate functional ethylenically unsaturated monomers may be prepared by the reaction of ethylenically unsaturated monomers containing 1,2- or 1,3-diols with phosgene, dialkylcarbonates, or cyclic carbonates.

Finally, although not preferred, cyclic carbonate functional ethylenically unsaturated monomers can be prepared by the thermal decomposition of ethylenically unsaturated monomers containing beta-hydroxy primary carbamates.

Monomer mixture (a) may further optionally comprise one or more additional ethylenically unsaturated monomers (aii) that are different from monomer (ai) and have one or more functional groups that are unreactive with the cyclic carbonate functional groups of monomer (ai) under oligomerization or polymerization conditions. That is, under free radical polymerization conditions as defined above, the functional groups of monomers (aii) will not react with the cyclic carbonate group of monomer (ai). In a most preferred embodiment, monomer mixture (a) will comprise one or more monomers (aii).

Monomer (aii) will be present in monomer mixture (a) in an amount of about 0 to about 99% by weight, based on the total weight of monomer mixture (a), more preferably about 30 to about 95% by weight, and most preferably about 50 to about 90% by weight, based on the total weight of monomer mixture (a).

Illustrative examples of such monomers (aii) include hydroxyl functional ethylenically unsaturated monomers, isocyanate functional ethylenically unsaturated monomers, carboxylic acid functional ethylenically unsaturated monomers, urea functional ethylenically unsaturated monomers, carbamate functional ethylenically unsaturated monomers and mixtures thereof, wherein ethylenically unsaturated monomers are as defined above. Preferred monomers (aii) are hydroxyl functional, acid functional, alkyl substituted, aryl substituted and isocyanate functional ethylenically unsaturated monomers

Illustrative hydroxyl functional ethylenically unsaturated monomers (aii) are hydroxyalkyl esters of acrylic acid or methacrylic acid such as hydroxyethylmethacrylate, hydroxypropylmethacrylate and mixtures thereof, with hydroxyethylmethacrylate being most preferred.

Illustrative isocyanate functional ethylenically unsaturated monomers (aii) include meta-isopropenyl-alpha,alpha-dimethylbenzyl isocyanate and isocyanatoethyl methacrylate. Meta-isopropenyl-alpha,alpha-dimethylbenzyl isocyanate is most preferred.

Illustrative carboxylic acid functional ethylenically unsaturated monomers (aii) are acrylic acid, methacrylic acid and mixtures thereof, with methacrylic acid being preferred.

Suitable urea functional ethylenically unsaturated monomers (aii) include allyl urea.

Ethylenically unsaturated monomers having carbamate functionality in the ester portion of the monomer may also be used as monomer (aii). Such monomers are well known in the art and are described, for example, in U.S. Pat. Nos. 3,479,328, 3,674,838, 4,126,747, 4,279,833, and 4,340,497, the disclosures of which are hereby incorporated by reference. For example, one method of synthesis involves reaction of a hydroxy ester with urea to form the carbamyloxy carboxylate (i.e., carbamate modified acrylate). Another method of synthesis reacts an alpha, beta-unsaturated acid ester with a hydroxy carbamate ester to form the carbamyloxy carboxylate. Additionally, the hydroxy group on a hydroxyalkyl carbamate can be esterified by reaction with acrylic or methacrylic acid to form a carbamate functional ethylenically unsaturated monomer. Other methods of preparing carbamate modified acrylic monomers are described in the art and can be utilized as well.

Monomer mixture (a) may further optionally comprise one or more nonfunctional ethylenically unsaturated monomers (aiii). Illustrative nonfunctional monomers (aiii) include vinyl monomers such as styrene, alpha-methyl styrene, vinyl toluene, tert-butyl styrene, and 2-vinyl pyrrolidone and alkyl esters of acrylic acid and/or methacrylic acid. Illustrative examples of alkyl esters of acrylic acid and/or methacrylic acid include ethyl (meth)acrylate, butyl (meth)acrylate, 2-ethylhexyl(meth)acrylate, cyclohexyl (meth)acrylate, lauryl (meth)acrylate, isodecyl (meth)acrylate, methyl (meth)acrylate.

Monomer (aiii) will be present in monomer mixture (a) in an amount of about 0 to about 99% by weight, based on the total weight of monomer mixture (a), more preferably about 30 to about 95, and most preferably about 50 to about 90, based on the total weight of monomer mixture (a).

Monomer mixture (a) will be polymerized under free radical or controlled polymerization conditions to provide the required cyclic carbonate functional starting material having an acrylic polymer as P with cyclic carbonate functional groups (bi). Most preferably, free radical polymerization processes will be used. Acrylic backbone polymer (b) may also comprise optional functional groups (bii) if monomer mixture (a) comprised optional monomers (aii).

Modified acrylics having the required two or more cyclic carbonate functional groups (bi) can also be used as the starting material. Such acrylics may be polyester-modified acrylics or polyurethane-modified acrylics, as is well known in the art. Polyester-modified acrylics modified with ε-caprolactone are described in U.S. Pat. No. 4,546,046 of Etzell et al, the disclosure of which is incorporated herein by reference. Polyurethane-modified acrylics are also well known in the art. They are described, for example, in U.S. Pat. No. 4,584,354, the disclosure of which is incorporated herein by reference. A non-limiting example of one such polymer is an acrylic resin made up of hydroxyethyl methacrylate, methyl methacrylate, and butyl acrylate which is then half capped with a diisocyanate such as isophorone diisocyanate to make an isocyanate functional polymer useful as material P. Cyclic carbonate groups may be incorporated into such modified acrylics via the reaction of oxirane groups and CO₂ to form cyclic carbonate groups as discussed above. Examples of suitable oxirane group-containing polymerizable monomers include, without limitation, glycidyl acrylate, glycidyl methacrylate, glycidyl crotonate, and allyl glycidyl ether.

Polyesters and ester oligomers having cyclic carbonate functional groups (bi) can also be used as the starting material in the method of the invention. It will be appreciated that P in this case is the polyester polymer or ester oligomer. Such polyesters are well-known in the art, and may be prepared by the polyesterification of organic polycarboxylic acids (e.g., phthalic acid, hexahydrophthalic acid, adipic acid, maleic acid) or their anhydrides with organic polyols containing primary or secondary hydroxyl groups (e.g., ethylene glycol, butylene glycol, 1,6-hexanediol, neopentyl glycol).

Cyclic carbonate groups may be incorporated into polyesters as follows. Polyesters, formed as described above, will generally have either hydroxy, acid or a mixture of both functionalities. Such functionalities can be used to provide the required cyclic carbonate groups (bi). For example, a hydroxy functional polyester may be reacted with a diisocyanate to form an isocyanate functional polyester. The reaction of this material with glycidol will form an epoxy functional polyester with internal urethane links with can then be reacted with CO2 to form cyclic carbonate groups. Polyesters that contain 1,2 or 1,3-diols can be converted into cyclic carbonate groups by reaction with carbonates such as dimethyl carbonate or diphenyl carbonate or by reaction with cyclic carbonates such as ethylene or propylene carbonate or by reaction with phosgene. Cyclic carbonate groups can also be incorporated by the reaction of acid or hydroxy groups on the polyester with respectively, allyl alcohol or vinyl acetic acid, followed by reaction with peroxide and then carbon dioxide.

Other functional polyesters useful as a starting material can be formed though the use of specialty capping alcohols and acids that are added during the polyester formation. For example, the addition of a hydroxy alkene followed by reaction with hydrogen peroxide will result in the placement of an epoxy group on the polyester. Reaction of this epoxy polyester with carbon dioxide will also result in the formation of a cyclic carbonate functional polyester.

Polyurethanes and urethane oligomers having required cyclic carbonate functional groups (bi) may also be used as the starting material. It will be appreciated that this instance, P is a polyurethane or urethane oligomer. Such starting materials can be prepared by a chain extension reaction of a polyisocyanate (e.g., hexamethylene diisocyanate, isophorone diisocyanate, MDI, etc.) and a polyol (e.g., 1,6-hexanediol, 1,4-butanediol, neopentyl glycol, trimethylol propane). Formulating with an appropriate amount of excess polyisocyanate will result in the polyurethane having free isocyanate functionality. Use of glycidol or 3-hydroxypropylene carbonate, for example, will functionalize the polyurethane with epoxy or cyclic carbonate groups respectively. As described above, epoxy groups can be converted into cyclic carbonate groups via reaction with CO₂.

Other compounds suitable for use as the starting material include those where P is a mono or polyfunctional compound such as nonchain-extended aliphatic, cycloaliphatic, an aromatic material which may or may not contain heteroatoms and which contain two or more cyclic carbonate groups (bi) or functional groups which may be converted into cyclic carbonate groups.

Examples of compounds suitable for use as the starting material include simple aliphatic functional materials such as erythritol bis-carbonate, monomeric diisocyanates like hexane diisocyanate, aliphatic polyamines such as 1,6-hexane diamine, anhydrides such as succinic anhydride, polyacids such as dodecane dioic acid, compounds having mixed functionality such as hydroxy pivalic acid, the like, and mixtures thereof. Aromatic functional materials may also be used such as 2,2-bis(4-hydroxyphenyl)propane. Suitable heteroatom functional materials include hydroxyneopentyl hydroxy pivalate. It will be appreciated that such compounds that do not have two or more cyclic carbonates thereon do contain functional groups that can be converted into the required two or more cyclic carbonate groups as described herein.

Other examples of suitable oligomers for use as the starting material include heterocyclic materials based on triazines and isocyanurates such as triamino triazine and tris-glycidyl isocyanurate.

Most preferred for use as a starting material herein are acrylic oligomers and polymers made using the (meth)acrylate esters of glycerine carbonate or 4-hydroxymethyl-1,3-dioxan-2-one, urethanes and acrylics functionalized using hydroxy cyclic carbonates such as glycerine carbonate, and isocyanurate based oligomers such as 1,3,5-triazine-2,4,6(1H,3H,5H)-trione.

On the average, at least one cyclic carbonate group (bi) of any starting material must undergo reaction with ammonia to produce a reaction product comprising material P having at least one, and most preferably at least two, gamma hydroxy primary carbamate groups.

The cyclic carbonate functional groups (bi) can be converted to the desired gamma hydroxy primary carbamates via reaction with ammonia. The reaction with ammonia will generally take place under mild conditions at temperatures from 0 to 60° C. It can be carried out in organic solvents such as methanol, or the reaction can be carried in water, or a mixture of water and organic solvents. When water is used as the sole solvent or as a part of a solvent blend, ammonium hydroxide may be used in place of ammonia. Alternatively, liquefied ammonia may be used as the solvent.

In one embodiment of the invention, optional functional groups (bii) of acrylic backbone polymer (b) may be reacted with one or more optional compounds (d) to provide a functional group (bii′). Functional group (bii) may act as a grafting site or as a precursor to a different functional group that was difficult to incorporate earlier. For example, a material P containing acid functional groups (bii) may be salted with a tertiary amine before the initiation of the ring opening reaction.

Compound (d) may be monomeric, oligomeric, or polymeric in nature, with monomeric being most preferred. It will be appreciated that compound (d) must have at least one functional group reactive with optional functional group (bii). The selection of compound (d) will be dependent upon the identity of both functional groups (bii) and (bii′). Illustrative compounds include those discussed below with respect to compounds (e).

The disclosed gamma hydroxyl primary carbamate functional materials may also be made via other processes.

For example, the gamma hydroxy primary carbamate functional compounds of the invention may be made by reacting a compound (a) and a compound (b).

Compound (a) will generally be one of the structures:

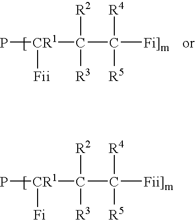

wherein all variable are as described above and functional groups Fi and Fii are separated by at least three carbon atoms, wherein said functional groups Fi and Fii are independently selected from the group consisting of functional groups convertible to primary carbamate groups. Preferred examples of functional groups Fᵢ and Fᵢᵢ convertible to primary carbamate groups are hydroxy groups and halide groups. Suitable halide groups include chloride, bromide, and iodide, with chloride being the most favored halide. Most preferably, functional groups Fi and Fii will be hydroxyl groups.

Suitable compounds (a) may include polyols, diols, polyhalides, and dihalides. However, the use of diols and dihalides as compound (a) is especially preferred as they are the most commercially available and economically feasible. Diols are most preferred for use as compound (a). Indeed, it is a particular benefit of the invention that it provides an economical and commercially feasible method of making thermally stable mono-carbamate compounds containing at least one functional group from compound (a) starting materials selected from the group consisting of dihalides and diols.

The selection of compound (b) is somewhat dependent upon the selection of functional groups Fᵢ and Fᵢᵢ of compound (a). In general, if functional group (i) is a hydroxyl group, it will be converted into a primary carbamate by reaction with a compound (b) selected from the group consisting of alkyl carbamates, cycloalkyl carbamates, ether carbamates, beta hydroxy alkyl carbamates, aryl carbamates, cyanic acid produced, for example, by the decomposition of urea, and phosgene followed by reaction with ammonia. If functional group (i) is a halide group, it may be converted into a primary carbamate group by reaction with a metal carbamate such as silver carbamate as discussed in P. Adams & F. Baron, “Esters of Carbamic Acid”, Chemical Review, v. 65, 1965. In a preferred embodiment, compound (b) will be selected from the group of alkyl carbamates, cycloalkyl carbamates, ether carbamates and aryl carbamates, and mixtures thereof, with alkyl carbamates being most preferred as compound (b).

Illustrative alkyl carbamates, cycloalkyl carbamates, and aryl carbamates include methyl carbamate, propyl carbamate, n-butylcarbamate, cyclohexyl carbamate, t-butyl carbamate, isopropyl carbamate, and phenyl carbamate. An example of a hydroxy alkyl carbamate is hydroxy ethyl carbamate. An example of an ether carbamate is 2-methoxyethyl carbamate. It will be appreciated that when (b) is selected from these compounds, reaction with suitable compounds (a) results in alcohols, phenols, ether alcohols and related materials as by-products. Examples of most preferred alkyl carbamates for use as compound (b) include methyl carbamate, isopropyl carbamate and n-butyl carbamate.

Compound (a) and compound (b) are reacted under conditions intended to minimize the formation of functional group Fᵢᵢ to a carbamate group. In general, compounds (a) and (b) will reacted under conditions such that no more than 10% of the functional group Fᵢᵢ is converted to a carbamate group, based on the starting amount of compound (a). More preferably, compounds (a) and (b) will be reacted under conditions such that no more than 5% of functional group Fᵢᵢ is converted to a carbamate group, and most preferably no more than 4% of functional group Fᵢᵢ will be converted to a carbamate group, all based on the starting amount of compound (a).

Thus, the formation of dicarbamate species is highly disfavored in this alternative method of making the disclosed materials. One technique to disfavor the formation of the dicarbamate is to use a deficit amount of compound (b), that is, the equivalent of the functional groups of compound (b) is less than the equivalent amount of functional group Fᵢ based on the starting amount of compound (a). In this case, the equivalent amount of compound (b) used in relationship to functional group Fᵢ can range from 0.99 to 1 to 0.25 to 1. An alternative technique that can be used to disfavor the formation of the dicarbamate when one or more than one equivalent of compound (b) are used in comparison to functional group Fᵢ on compound (a) is to stop the reaction before all of functional Fᵢ is converted to a primary carbamate. This second technique works best for reaction conditions that have a high degree of selectivity such as transcarbamation reactions. In comparison, this technique would be disfavored in a more nonselective reaction such as that between a hydroxy group and isocyanic acid.

While not wishing to be bound to a particular theory, it is believed that the effectiveness of these two approaches can be increased by increasing the relative degree of steric hindrance surrounding functional groups Fᵢ and Fᵢᵢ on compound (a). That is, in general, dicarbamate formation can be diminished if the degree of steric hindrance surrounding functional group Fᵢᵢ is greater than the degree of steric hindrance on functional group Fᵢ. This relationship is believed to hold true regardless of the method of reaction selected.

The disclosed gamma hydroxy primary carbamate materials may be considered to be of the formula:

(C_{NON-CARBAMATE FUNCTIONAL GROUPS})ᵢ-P—(C_{NH3})ⱼ

wherein P is a material, oligomer, or polymer as discussed above, C_{NON-CARBAMATE FUNCTIONAL GROUPS}) are any noncarbamate functional groups, C_{NH3} is the reaction product of ammonia with a cyclic carbonate functional group (bii), and i and j represent the total number of both functional groups.

P is defined as above except that in this formula, P may, but is not required to, contain two or more cyclic carbonate groups (bi) but may further comprise additional functional groups that are inert with respect to cyclic carbonate functional groups under the ring opening reaction conditions.

Suitable examples of C_{NON-CARBAMATE FUNCTIONAL GROUPS} include hydroxy, acid, ethylenically unsaturated groups, polyesters, simple alkyl groups, aromatic groups, and the like and mixtures thereof.

i and j represent the total number of the respective functional groups and may be the same or different, but will most preferably be different. i is a number from 0 to about 49, preferably from 1 to 20 and most preferably from 1 to 10, while j is a number from 1 to about 50, about 2 to about 30 and most preferably from about 2 to about 10.

C_{NH3} is the reaction product of ammonia with the cyclic carbonate functional group (bi) described above and will comprise one or more structures of the formula:

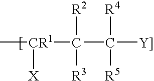

wherein X and Y are either a primary carbamate group or a hydroxyl group but may not be the same, and R¹, R², R³, R⁴, and R⁵ are each at least one of H, an alkyl group, a heteroatom containing group or mixtures thereof. In a most preferred embodiment, R¹, R², R¹, R⁴, and R⁵ will each be H.

In a particularly preferred embodiment of the invention, P will be an acrylic oligomer or polymer. In this embodiment, the gamma hydroxy primary carbamate acrylic multifunctional materials of the invention will have the formula:

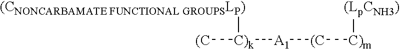

wherein A is the residue resulting from the polymerization of an ethylenically unsaturated monomer which does not contain a cyclic carbonate group but may contain a functional group (bii), L is a linking group, p is number of from 0 to 5, C_{NONCARBAMATE FUNCTIONAL GROUPS} is as defined above, C_{NH3} is the reaction product of ammonia with a cyclic carbonate functional group as discussed above, and k, l, and m represent the total number of monomers or repeating units. It will be appreciated that in the above formula, the bond connecting L to the ethylenically unsaturated backbone is not attached to either carbon but is depicted as somewhere in the middle to illustrate the two possible isomers.

Ethylenically unsaturated monomers suitable for providing repeating units A may be as described above with respect to ethylenically unsaturated monomers (aii) and (aiii).

L is a linking group selected from aliphatic groups, cycloaliphatic groups, aromatic groups and mixtures thereof of from one to seven carbons. L may contain heteratoms such O, N, S, and mixtures thereof and/or functional groups such as esters, ethers, urethanes, ureas, amides, and mixtures thereof as discussed above. Preferred groups suitable for use as linking group L are esters and urethanes, with esters being most preferred.

p is number of from 0 to 5, with numbers of from 1 to 5 being preferred and 1 being most preferred. It will thus be appreciated that L is an optional linking group but one which will preferably be present.

C_{NONCARBAMATE} and C_{NH3} are as defined above.

k, L, and m represent the total number of monomers comprising the desired β or higher hydroxy primary carbamate functional acrylic polymer or oligomer of the invention. k is from 1 to 95% of the total sum of k, L, and m, preferably from 5 to 80, and most preferably from 20 to 50, based on the total sum of k, 1, and m. l is from 0 to 98% of the total sum of k, 1, and m, preferably from 30 to 95%, and most preferably from 50 to 90%, based on the total sum of k, l, and m. m is from 1 to 95% of the total sum of k, l, and m, preferably from 5 to 80 and most preferably from 20 to 60, based on the total sum of k, l, and m

When the method of the invention is used to provide waterborne gamma hydroxy primary carbamate functional materials, the values of i and j in the formula: (C_{NONCARBAMATE})i-P-(C_{NH3})j may be useful in predicting the degree of water solubility. That is, as noted above, materials useful in waterborne compositions may range from completely water-soluble to those which are relatively insoluble but which are stabilized in water by the formation of micelles via dispersible functional groups. As noted above, i and j represent the total number of the respective functional groups.

Combined with number average molecular weight of P, the values of i and j may be used as a guide to predict the water dispersibility or solubility of waterborne gamma hydroxy primary carbamate functional materials. For example, when the result of dividing the molecular weight of material P by the sum of i+j is between 500 and 2000, and when the result of dividing the molecular weight of material P by just i is between 320 to 1000, the gamma hydroxy primary carbamate functional materials invention may be suitable for use in electrodeposition coating compositions wherein the multifunctional material is initially dispersible in waterborne but precipitates out upon the introduction of an electrical current.

Alternatively, when the result of dividing the molecular weight of material P by the some of i+j is between 400 and 800, and the result of dividing the molecular weight of material P by i is between 450 to 1500, the gamma hydroxy primary carbamate functional materials of the invention may be described as suitable for use in aqueous dispersions.

Finally, when the result of dividing the molecular weight of material P by the sum of i+j is less than 600, and the result of dividing the molecular weight of material P by i is between 320 to 2500, the waterborne multifunctional materials of the invention may be described as materials which are substantially soluble in water. However, it will be appreciated that the behavior of the multifunctional materials in water is also dependent upon the nature of material P as well as the polar/ionic nature of graft moiety (cii).

More precisely, it may be stated that the water dispersibility and/or solubility of the gamma hydroxy primary carbamate functional materials of the invention may be identifiable based on two values calculated using i, j, and the number average molecular weight of P (P_{MW}), i.e., WV₁ and WV₂ wherein

WV₁=PMW÷(i+j) and WV₂=P_{MW}÷(i).

It may be generally stated that waterborne gamma hydroxy primary carbamate functional materials of the invention will be electrodepositable if WV₁ is a number from 500 to 2000 and WV₂ is a number from 320 to 1000; water dispersible if WV₁ is a number from 400 to 800 and WV₂ is a number from 450 to 1500; and water soluble if WV₁ is a number less than 600 and WV₂ is a number from 320 to 2500.

The relative ratio of i and j needed to achieve a set level of water solubility is dependent on the make up of P, any non-gamma hydroxy primary carbamate functional groups and the overall molecular weight, and must be determined on a case-by-case bases. However, the foregoing values are illustrative of preferred embodiments.

The gamma hydroxy primary carbamate functional materials of the invention are particularly suitable for use in automotive coating compositions, especially electrodeposition coatings, primers, topcoats, basecoats, and/or clearcoats, with clearcoats being especially preferred. The resultant reactive compound may be used in a variety of compositions used in film-forming applications, including but not limited to curable coating compositions, sealant compositions, and adhesive compositions. The disclosed gamma hydroxy primary carbamate functional materials may be used as a film-forming component of such compositions, as a reactive diluent that may replace some or all traditional solvents, or may alternatively be used as a reactant to form polymeric and oligomeric components for use in such compositions, or combinations thereof.

Coating compositions comprising the gamma hydroxy primary carbamate functional materials preferably form the outermost layer or layer of coating on a coated substrate. Preferably, the instant coating compositions are applied over one or more layers of primer coatings. For example, the coating compositions of the invention may be used as an automotive topcoat coating applied over a layer of electrocoat primer and/or primer surfacer.

When such coating compositions are used as topcoat coatings, they preferably have a 20 degree gloss, as defined by ASTM D523-89, of at least 80 or a DOI, as defined by ASTM E430-91, of at least 80, or both. Such gloss and DOI are particularly useful in providing an automotive finish that will appeal to the buyer of the vehicle. Topcoat coatings may be one coat pigmented coatings or may be a color-plus-clear composite coating.

Coating compositions comprising the gamma hydroxy primary carbamate functional materials of the present invention, if used as a one coat pigmented coating or the color coating of a color-plus-clear composite coating, will include one or more pigments well-known in the art, such as inorganic pigments like titanium dioxide, carbon black, and iron oxide pigments, or organic pigments like azo reds, quinacridones, perylenes, copper phthalocyanines, carbazole violet, monoarylide and diarylide yellows, naphthol orange, and the like.

In a preferred embodiment, the coating compositions comprising the gamma hydroxy primary carbamate functional materials of the present invention will be clearcoats used in color-plus-clear composite coatings. Such clearcoats may be applied over a color coat according to the invention or may, alternatively, be applied over a color coat of a formulation already known in the art. Pigmented color coat or basecoat compositions for such composite coatings are well known in the art and do not require explanation in detail herein. Polymers known in the art to be useful in basecoat compositions include acrylics, vinyls, polyurethanes, polycarbonates, polyesters, alkyds, and polysiloxanes. Such basecoats may comprise the acrylic graft copolymer of the invention. Preferred polymers include acrylics and polyurethanes, especially the gamma hydroxy primary carbamate functional materials of the invention. In one preferred embodiment of the invention, the basecoat composition also utilizes gamma hydroxy primary carbamate functional materials of the invention.

Curable coating compositions comprising the gamma hydroxy primary carbamate functional materials of the invention will be crosslinkable and will thus comprise one or more type of crosslinking agents having one or more crosslinkable functional groups. Such groups include, for example, hydroxy, isocyanate, amine, epoxy, acrylate, vinyl, silane, and acetoacetate groups. These groups may be masked or blocked in such a way so that they are unblocked and available for the cross-linking reaction under the desired curing conditions, generally elevated temperatures. Useful crosslinkable functional groups include hydroxy, epoxy, acid, anhydride, silane, and acetoacetate groups. Preferred crosslinking agents will have crosslinkable functional groups that include hydroxy functional groups and amino functional groups and isocyanate groups. Di- and/or polyisocyanates and/or aminoplast resins are most preferred for use as crosslinking agents in coating compositions comprising the acrylic graft polymer of the invention. Other mixed crosslinkers may also be used.

For example, basecoat-coating compositions comprising the gamma hydroxy primary carbamate functional materials of the invention may require two or more separate crosslinking agents in order to react with both the primary carbamate group and the hydroxyl group. For example, the crosslinking agent may be one or more of an aminoplast resin, a polyisocyanate or a blocked polyisocyanate resin (including an isocyanurate, biuret, or the reaction product of a diisocyanate and a polyol having less than twenty carbon atoms), an acid or anhydride functional crosslinking agent or a mixture thereof.

Other materials well-known to the coatings artisan, for example, surfactants, fillers, stabilizers, wetting agents, dispersing agents, adhesion promoters, UV absorbers, light stabilizers such as HALS, antioxidants, solvents, catalysts, and/or rheology control agents, may be incorporated into the coating composition. The amount of these materials used must be controlled to achieve the desired performance properties and/or to avoid adversely affecting the coating characteristics.

It will be appreciated that suitable solvents include organic solvents, water, water-soluble solvents, and mixtures thereof. It will be appreciated that solvent borne coating may comprise minor amounts of water while waterborne coatings such as electrodeposition coatings may comprise organic solvents.

Coating compositions can be coated onto an article by any of a number of techniques well known in the art. These include, for example, spray coating, dip coating, roll coating, curtain coating, and the like. For automotive body panels, spray coating is preferred. When the coatings will be relatively thick, they are usually applied in two or more coats separated by a time sufficient to allow some of the water and/or solvent evaporate from the applied coating layer (“flash”). The coats as applied are usually from 1 to 3 mils of the coating composition, and a sufficient number of coats are applied to yield the desired final coating thickness.

Where a color-plus-clear composite coating is applied to the prepared substrate, the color coat is usually applied in one or two coats, allowed to flash, and the clear coat is then applied to the uncured color coat in one or two coats. The two coating layers are then cured simultaneously. Preferably, the cured base coat layer is 0.5 to 1.5 mils thick and the cured clear coat layer is 1 to 3 mils, more preferably 1.6 to 2.2 mils thick.

Coating compositions comprising the gamma hydroxy primary carbamate functional materials of the invention are preferably subjected to conditions so as to cure the coating layers. Although various methods of curing may be used, thermal curing is preferred. Generally, thermal curing is effected by exposing the coated article to elevated temperatures provided primarily by radiative heat sources. Curing temperatures will vary depending on the particular blocking groups used in the crosslinking agents, however they generally range between 93 degree C. and 177 degree C. In a preferred embodiment, the cure temperature is between 135 degree C. and 165 degree C. In another preferred embodiment, a blocked acid catalyst is included in the composition and the cure temperature is between 115 degree C. and 140 degree C. In a different preferred embodiment, an unblocked acid catalyst is included in the composition and the cure temperature is between 80 degree C. and 100 degree C. The curing time will vary depending on the particular components used and physical parameters, such as the thickness of the layers. Typical curing times range from 15 to 60 minutes, and preferably 15-25 minutes at the target temperature.

### EXAMPLES

### Example 1a

### Prophetic

### Preparation of 1,3-dioxan-2-one, 5-methacrylate

A mixture of 30 parts of anhydrous amyl acetate and 30.4 parts of 5-hydroxy-1,3-dioxan-2-one is heated to 120° C. under a modified water free atmosphere made up of 95 parts nitrogen and 5 parts oxygen. Then 39.6 parts of methacrylic acid is slowly added. Once the addition is complete the reaction is kept at 120° C. until all of the anhydride is opened, as determined by IR spectrometry. The solvent and newly made methacrylic acid are then removed by high vacuum distillation.

### Example 1b Prophetic

### Making a Polymer of 1,3-dioxan-2-one, 5-methacrylate

30 parts of amyl acetate is heated to 130° C. under an inert atmosphere. Then a mixture of 40 parts of 1,3-dioxan-2-one, 5-methacrylate from Example 1a, 10 parts of 2-carbamate ethyl methacrylate, 5 parts of ethyl acrylate and 5 parts of t-butyl peroctoate is added over a four hour period. Once the addition is complete a mixture of 5 parts of amyl acetate and 0.5 parts t-butyl peroctoate is added over a thirty-minute period. Then 4.5 parts of amyl acetate are added and the reaction mixture is held for one hour at 130° C.

### Example 1c Prophetic

### Ring Opening of a Polymer of 1,3-dioxane-2-one, 5-methacrylate

A mixture of 70 parts of the polymer made from Example 1b and 30 parts of methanol are placed in a flask equipped with a cooling coil. Then ammonia gas is slowly bubbled into the solution. The reaction temperature is kept below 40° C. Once all of the cyclic carbonate groups are ring opened to form gamma hydroxy primary carbamate groups the excess ammonia and solvent are removed by vacuum distillation. The solvent free resin is then dissolved into 30 parts DI water.

### Example 2

### Making a Gamma Hydroxy Primary Carbamate Urethane

A mixture of 43.5 parts of the isocyanurate of 1,6-hexane diisocyanate, 0.07 parts of dibutyl tin dilaurate and 30 parts of anhydrous methyl propyl ketone are heated under an inert atmosphere to 70° C. Then 37.9 parts of 5-hydroxy-1,3-dioxan-2-one is slowly added. Once the reaction is complete, the reaction mixture is cooled to room temperature and 30 marts of methanol are added. Ammonia gas is then slowly bubbled into the reaction mixture. Once all of the cyclic carbonate groups have been transformed into gamma hydroxy carbamate groups the excess ammonia and solvent are removed by vacuum distillation. The resulting material is then dissolved into 40 parts of DI water.

### Example 3

### Making a Gamma Hydroxy Primary Carbamate Urethane

A mixture of 33.1 parts of hexane diisocyanate, 0.7 parts of dibutyl tin dilaurate and 30 parts of anhydrous methyl propyl ketone are heated under an inert atmosphere to 60° C. Then 21.3 parts of 5,5-bis(hydroxymethyl)-1,3-dioxan-2-one is slowly added. After the addition is complete, the reaction mixture is held at 60° C. for one hour. Then 15.6 parts of hydroxy propyl carbamate is added. Once the reaction is complete, the reaction mixture is cooled to room temperature and 30 parts of methanol are added. Ammonia gas is then slowly bubbled into the reaction mixture. Once all of the cyclic carbonate groups have been transformed into gamma hydroxy carbamate groups the excess ammonia and solvent are removed by vacuum distillation. The resulting material is then dissolved into 40 parts of DI water.

I claim:

1. A gamma hydroxy primary carbamate comprising one or more structures of the formula: 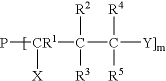
wherein
X and Y are either a primary carbamate group or a hydroxyl group but are not the same,
m is a number from 2 to 50,
R¹, R², R³, R⁴, and R⁵ are each at least one of H, an alkyl group, a heteroatom containing group, or mixtures thereof, and
P is at least one hydrocarbon based member selected from a compound, an oligomer or polymer having more than 6 carbon atoms.

2. The gamma hydroxy primary carbamate of claim 1 wherein X is a primary carbamate group.

3. The gamma hydroxy primary carbamate of claim 1 wherein X is a hydroxyl group.

4. The gamma hydroxy primary carbamate of claim 1 wherein m is at least 3.

5. The gamma hydroxy primary carbamate of claim 1 wherein m is at least 4.

6. The gamma hydroxy primary carbamate of claim 1 wherein m is about 4 to about 30.

7. The gamma hydroxy primary carbamate of claim 1 wherein each of R¹, R², R³, R⁴, and R⁵ are H.

8. The gamma hydroxy primary carbamate of claim 1 wherein one or more of R¹, R², R³, R⁴, and R⁵ are selected from alkyl groups having from 1 to 4 carbons.

9. The gamma hydroxy primary carbamate of claim 8 wherein one or more of R¹, R², R³, R⁴, and R⁵ are selected from alkyl groups having from 1 to 2 carbons.

10. The gamma hydroxy primary carbamate of claim 1 wherein each of R¹, R², R³, R⁴, and R⁵ are free of any heteroatom containing groups.

11. The gamma hydroxy primary carbamate of claim 1 wherein P is a compound.

12. The gamma hydroxy primary carbamate of claim 1 wherein P is a polymer or oligomer.

13. The gamma hydroxy primary carbamate of claim 12 wherein P is a polymer.

14. The gamma hydroxy primary carbamate of claim 13 wherein P is a polymer selected from the group consisting of acrylic polymers, polyurethane polymers, and polyester polymers.

15. The gamma hydroxy primary carbamate of claim 14 wherein P is an acrylic polymer.

16. The gamma hydroxy primary carbamate of claim 15 wherein P comprises heteroatom containing groups.

17. The gamma hydroxy primary carbamate of claim 16 wherein P comprises on average per molecule, one or more heteroatom containing groups selected from the group consisting of acid groups, ester groups, amide groups, ether groups, urethane groups, epoxy groups, hydroxyl groups, and mixtures thereof.

18. A film-forming composition comprising
(I) a gamma hydroxy primary carbamate comprising one or more structures of the formula: 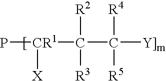
wherein
X and Y are either a primary carbamate group or a hydroxyl group but are not the same,
m is a number from 1 to 50,
R¹, R², R³, R⁴, and R⁵ are each at least one of H, an alkyl group, a heteroatom containing group or mixtures thereof, and
P is at least one hydrocarbon based member selected from a compound, an oligomer or polymer having more than 6 carbon atoms, and
(II) a crosslinking component having one or more functional groups reactive with at least one of X or Y of gamma hydroxy primary carbamate functional compound (I).

19. The film-forming composition of claim 18 comprising the gamma hydroxy primary carbamate wherein m is at least 4.

20. The film-forming composition of claim 19 comprising the gamma hydroxy primary carbamate wherein m is about 4 to about 30.

21. The film-forming composition of claim 18 comprising a crosslinking component selected from the group consisting of aminoplasts, isocyanate functional crosslinking agents, blocked isocyanate functional crosslinking agents, acid functional crosslinking agent, anhydride functional crosslinking agent, epoxy functional crosslinking agent, and mixtures thereof.

22. The film-forming composition of claim 18 comprising a crosslinking component selected from the group consisting of aminoplasts, isocyanate functional crosslinking agents, blocked isocyanate functional crosslinking agents, and mixtures thereof.

23. A method of making a gamma hydroxy primary carbamate functional material, comprising
providing a starting material comprising two or more cyclic carbonate groups (bi) and of the structure: 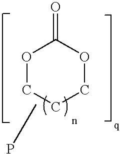
wherein n is 1, q is a number from 2 to 50, and P is a hydrocarbon based material selected from the group consisting of compounds, oligomers, and polymers having more than 6 carbon atoms, and
reacting at least one cyclic carbonate functional group (bi) with ammonia to provide a gamma hydroxy primary carbamate group of the structure: 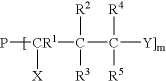
wherein X and Y are either a primary carbamate group or a hydroxyl group but may not be the same, m is a number from 2 to 50, R¹, R², R³, R⁴, and R⁵ are each at least one of H, an alkyl group, a heteroatom containing group, or mixtures thereof, and P is at least one hydrocarbon based member selected from a compound, an oligomer or polymer having more than 6 carbon atoms.

24. The method of claim 23 wherein q is at least 4.
